(19) United States
(12) Patent Application Publication
(10) Pub. No.: US 2026/0318893 A1
(43) Pub. Date: Oct. 15, 2026

(54) QUANTIFYING DIFFUSION OF METHANE GAS PLUMES

(71) Applicant: The Board of Regents of the University of Oklahoma, Norman, OK (US)

(72) Inventors: Xiao-Ming Hu, Norman, OK (US); Wesley T. Honeycutt, Norman, OK (US)

(21) Appl. No.: 19/311,677

(22) Filed: Nov. 4, 2025

Related U.S. Application Data
(60) Provisional application No. 63/687,771, filed on Aug. 27, 2024.

(51) Int. Cl. G01N 33/00

(52) U.S. Cl. CPC G01N 33/0047; G01N 33/0031; G01N 33/007

(57) ABSTRACT

A method for evaluating a plume from a fugitive gas emission includes the steps of detecting the fugitive gas emission, obtaining weather information for the area of interest surrounding the detected fugitive gas, and using a computer-implemented model to estimate the size and concentration of the gas plume based on the detected concentrations of the fugitive gas and the observed weather conditions. Suitable models include the WRF-GHG model deployed in a large eddy simulation mode with grid spacing in a range of about 5 meters to about 1 km. The model plume can be examined to determine the likely source of the fugitive emission or the quantity of gas emitted over time through the plume.

### CROSS REFERENCE TO RELATED APPLICATIONS

This application claims the benefit of U.S. Provisional Patent Application Ser. No. 63/687,771 field Aug. 27, 2024 and entitled “Quantifying Diffusion of Methane Gas Plumes,” the disclosure of which is incorporated by reference as if fully set forth herein.

### STATEMENT OF GOVERNMENT SUPPORT

This invention was made with government support under Contract Number DE-FE0032292 awarded by the Department of Energy. The government has certain rights in the invention.

### BACKGROUND

Methane (CH4) is a potent greenhouse gas (GHG) that contributes significantly to global climate warming. Methane is responsible for around 30% of the effective radiative forcing from anthropogenic emissions of greenhouse gases and their precursors. Methane is attributable to about 0.6 °C increase in global surface air temperature during the industrial era (1750-2019). As one of the major sectors for CH4 emissions, the oil and gas industry was estimated to contribute around 56% (67 Tg) of CH₄ of annual global anthropogenic CH4 emissions during 2010-2019 in the latest global methane budget. Ultra-emitters, with short-term leak events with high CH4 release (>25 tons/hour), could contribute an additional 8 Tg of CH₄ annually.

Nevertheless, large uncertainties still exist in the current estimation of these emissions on both regional and national scales. This is mainly due to the lack of consistency in emission accounting methods and emission factors. Methane can be emitted both intentionally and unintentionally during the operation, maintenance, and system disruptions of the oil and gas industry. In addition to the potential climate and environmental benefits, reducing methane emissions can also bring significant economic values. A report by the ICF International in 2014 estimated that a 40% reduction in CH4 emissions from the U.S. onshore oil and gas industry would lead to $100 million saved annually. However, implementing emission reduction measures would require initial investments of $2.2 billion. This highlights the challenges that oil and gas industry is facing. Notably, approximately 30% of the U.S. domestic primary energy production is ensconced in Southern Great Plains, including Texas and Oklahoma. The economies of these states depend heavily on energy production. For example, 8.2% of employment in Oklahoma is directly related to energy extraction with the majority remaining non-farm jobs indirectly dependent on this industry. Oklahoma provides economic incentives to reduce methane emissions from these energy production operations. Apart from the environmental ramifications, there is a cost benefit from keeping methane gas properly contained in gathering systems, transportation lines, and processing facilities. The annual cost estimate for leak detection and repair for a facility averaged across all facilities was estimated to be $191,075 in 2014, a sizeable outlay for maintenance of equipment that is often remote and difficult to monitor. A reduction in monitoring frequency through targeted application of resources and refined accuracy through improved background monitoring would reduce this cost, and thereby reduce the barrier to attainment.

The identification of sources for preventing or mitigating CH₄ emissions is often hampered by the lack of CH₄ detection with adequate accuracy and spatiotemporal coverage, and the absence of accurate CH₄ forward modelling used in top-down inversion systems. CH₄ sensors could be either too expensive for wide deployment or have too low precision to detect ambient CH₄ enhancements. Currently, the EPA leak detection and repair (LDAR) protocols rely heavily on optical gas imaging (OGI) cameras. While this technology is useful for identifying leak locations, it is limited to monitoring nearby sources with CH₄ enhancements on the order of 5000 ppm-m when the temperature difference between gas and background is less than 2 °C. The operation of OGI cameras is subject to environmental conditions because OGI cameras require sunlight and a temperature difference between the fugitive gas with the background environment. These limitations prevent the successful use of OGI cameras in nocturnal or low-temperature conditions. For example, in colder periods it takes longer for the sun to heat the ground, which may lead to failure of OGI cameras.

CH₄ plumes are highly dictated by winds and atmospheric boundary layer (ABL) structure and associated turbulence fields. Given the same emission rate, CH₄ plumes may present significantly different characteristics (e.g., concentration and plume length) in different boundary layer conditions. During morning and evening transition hours, the boundary layer experiences profound changes. Different boundary layer stability conditions and temperature profiles may result in different types of CH₄ plumes, such as fanning, looping, lofting, fumigating, or trapping. Assuming each of these conditions may happen under the same emission rate, an OGI camera observation occurring beyond the fence or property line at an oil & gas well pad emitting fugitive CH₄ may appear drastically different from the same observation point when operating under standard EPA Method 21 protocols because the plume may be either dispersed or concentrated. Use of OGI detection is principally qualitative since attempting to quantify such a plume without strong information about the boundary layer increases the likelihood of error. OGI cameras are incapable of quantitatively assessing the mass flow rates of leaks. Furthermore, CH₄ plumes in a changing boundary layer are hard to accurately simulate in forward simulations, which prevents an accurate top-down emission inversion in addition to measurement error. Some GHG gas inversion systems used a simple Gaussian plume model, which poorly considers the response of dynamics of plumes to atmospheric boundary layer processes because the Gaussian plume model assumes a uniform flow with homogeneous and stationary turbulence which is rarely valid in a real atmosphere.

Numerical weather prediction (NWP)-based GHG modelling provides a promising alternative to simulate the GHG plumes in real atmosphere environments. The Weather Research and Forecasting (WRF) model with Chemistry (WRF Chem) is often used for regional simulation of GHGs using grid spacings of a few kilometers or sub-kilometers. These simulations often use GHG emissions aggregated in grid cells as coarse as one kilometer based on bottom-up inventories, and they can only simulate GHG plumes at urban or basin scales. Fugitive or vented emissions from single points are often not available from inventory data. Thus, conventional WRF-Chem simulations cannot resolve CH4 plumes from individual sites, which may only spread or span for a few kilometers or even a few hundred meters and occur every few kilometers, let alone individual oil and gas wells spaced a few meters apart in modern horizontally drilled facilities. Lack of high-resolution NWP-based simulation of fugitive plumes is partially due to the lack of ground-based measurements for model validation. Most in situ GHG measurement networks are designed to measure aggregated urban emissions rather than individual fugitive plumes.

There is, therefore, a need for systems and methods for accurately modeling plumes for fugitive and vented GHG emissions. It is to these and other objectives that the disclosed embodiments are directed.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein. The figures are not necessarily to scale and certain features and certain views of the figures may be shown as exaggerated in scale or in schematic in the interest of clarity and conciseness. The patent or application file contains at least one drawing executed in color. Copies of this patent or patent application publication with color drawing(s) will be provided by the Office upon request and payment of the necessary fee.

FIG. 1A shows land elevations over the fourth domain, taken from the Shuttle Radar Topography Mission (SRTM) data at a resolution of 1 arc-second (30 meters), illustrating the release site and driving paths for the experimental region.

FIG. 1B shows satellite images of the experimental region surrounding the release site, with driving paths marked. The experimental region shows a short red route and a long blue route, the west-east portion of which is 240 m and 1.45 km to the north of the site, respectively.

FIG. 2 shows controlled CH₄ release rate at the release site on a single test date.

FIG. 3A shows simulated CH₄ plumes at the release site in domain 4 overlaid with observation during sunrise.

FIG. 3B shows simulated CH₄ plumes at the release site in domain 4 overlaid with observation during noon.

FIG. 3C shows simulated CH₄ plumes at the release site in domain 3 overlaid with observation during sunrise.

FIG. 3D shows simulated CH₄ plumes at the release site in domain 3 overlaid with observation during noon.

FIG. 4 shows a time series of observed CH₄ enhancement at 250 m from the release site with the color shade in the background depicting the time-based shift from morning to afternoon.

FIG. 5A provides the surface temperature at 2 m above the ground (T2) and simulated PBL height (PBLH) at the release site.

FIG. 5B displays wind vectors at the MADIS site in Pampa, TX (35.6 N, 100.98 W) on the date of the release observed and simulated in the inner most domain with a large eddy simulation (LES) configuration. A reference vector of 3 m/s is marked in FIG. 5B.

FIG. 6A shows an observed and simulated profile of wind speed at Amarillo, TX (75 km to the southwest of the release site) at 0700 local time on the test date.

FIG. 6B shows an observed and simulated profile of potential temperature at Amarillo, TX (75 km to the southwest of the release site) at 0700 local time on the test date.

FIG. 6C shows an observed and simulated profile of water vapor mixing ratio at Amarillo, TX (75 km to the southwest of the release site) at 0700 local time on the test date.

FIG. 6D shows an observed and simulated profile of wind speed at Amarillo, TX (75 km to the southwest of the release site) at 1900 local time on the test date.

FIG. 6E shows an observed and simulated profile of potential temperature at Amarillo, TX (75 km to the southwest of the release site) at 1900 local time on the test date.

FIG. 6F shows an observed and simulated profile of water vapor mixing ratio at Amarillo, TX (75 km to the southwest of the release site) at 1900 local time on the test date.

FIG. 7A shows horizontal spatial distributions of CH₄ plumes during sunrise.

FIG. 7B shows horizontal spatial distributions of CH₄ plumes during late morning.

FIG. 7C shows vertical cross-sections of CH₄ plumes during sunrise.

FIG. 7D shows vertical cross-sections of CH₄ plumes during late morning.

FIG. 8A shows vertical cross-sections of mixing coefficients during sunrise.

FIG. 8B shows vertical cross-sections of mixing coefficients during late morning.

FIG. 8C shows potential temperatures during sunrise.

FIG. 8D shows potential temperatures during late morning.

FIG. 9A shows horizontal spatial distributions of temperature overlaid with horizontal wind vectors during sunrise.

FIG. 9B shows horizontal spatial distributions of temperature overlaid with horizontal wind vectors during late morning.

FIG. 9C shows vertical wind speed overlaid with horizontal wind vectors during sunrise.

FIG. 9D shows vertical wind speed overlaid with horizontal wind vectors during late morning.

FIG. 10A shows horizontal spatial distributions of CH₄ enhancement plumes overlaid with wind vectors at 11:30.

FIG. 10B shows horizontal spatial distributions of CH₄ enhancement plumes overlaid with wind vectors at 11:45.

FIG. 10C shows horizontal spatial distributions of CH₄ enhancement plumes overlaid with wind vectors at 12:35.

FIG. 10D shows vertical cross-section of CH₄ enhancement plumes overlaid with wind vectors at 11:30.

FIG. 10E shows vertical cross-section of CH₄ enhancement plumes overlaid with wind vectors at 11:45.

FIG. 10F shows vertical cross-section of CH₄ enhancement plumes overlaid with wind vectors at 12:35.

FIG. 11A shows horizontal spatial distribution of surface temperature at T2 overlaid with horizontal wind vectors at 11:30.

FIG. 11B shows horizontal spatial distribution of surface temperature at T2 overlaid with horizontal wind vectors at 11:45.

FIG. 11C shows horizontal spatial distribution of surface temperature at T2 overlaid with horizontal wind vectors at 12:35.

FIG. 11D shows horizontal spatial distribution of vertical wind speed at 60 m AGL overlaid with horizontal wind vectors at 11:30.

FIG. 11E shows horizontal spatial distribution of vertical wind speed at 60 m AGL overlaid with horizontal wind vectors at 11:45.

FIG. 11F shows horizontal spatial distribution of vertical wind speed at 60 m AGL overlaid with horizontal wind vectors at 12:35.

FIG. 12A depicts mixing ratios in the west-to-east direction along the short route (250 m north of the release site) observed and simulated using three subgrid-scale (SGS) mixing closures, i.e., KM2 (1.5 order TKE closure) in domain 4.

FIG. 12B depicts mixing ratios in the west-to-east direction along the short route (250 m north of the release site) observed and simulated using three subgrid-scale (SGS) mixing closures, i.e., KM 3 (first-order Smagorinsky closure) in domain 4.

FIG. 12C depicts mixing ratios in the west-to-east direction along the short route (250 m north of the release site) observed and simulated using three subgrid-scale (SGS) mixing closures, i.e., KM5 (scale-aware 3D-TKE scheme) in domain 4.

FIG. 12D depicts mixing ratios in the west-to-east direction along the short route (250 m north of the release site) observed and simulated using three subgrid-scale (SGS) mixing closures, i.e., KM2 (1.5 order TKE closure) in domain 3.

FIG. 12E depicts mixing ratios in the west-to-east direction along the short route (250 m north of the release site) observed and simulated using three subgrid-scale (SGS) mixing closures, i.e., KM 3 (first-order Smagorinsky closure) in domain 3.

FIG. 12F depicts mixing ratios in the west-to-east direction along the short route (250 m north of the release site) observed and simulated using three subgrid-scale (SGS) mixing closures, i.e., KM5 (scale-aware 3D-TKE scheme) in domain 3.

FIG. 13A illustrates the vertical cross-section of simulated vertical mixing coefficient (KHV) using SGS mixing closure KM2.

FIG. 13B illustrates the vertical cross-section of simulated vertical mixing coefficient (KHV) using SGS mixing closure KM3.

FIG. 13C illustrates the vertical cross-section of simulated vertical mixing coefficient (KHV) using SGS mixing closure KM5.

FIG. 13D illustrates the vertical cross-section of potential temperature using SGS mixing closure KM2.

FIG. 13E illustrates the vertical cross-section of potential temperature using SGS mixing closure KM3.

FIG. 13F illustrates the vertical cross-section of potential temperature using SGS mixing closure KM5.

FIG. 14A depicts mixing ratios along the short route observed and simulated using SGS mixing closure KM2 in domain 4.

FIG. 14B depicts mixing ratios along the short route observed and simulated using SGS mixing closure KM3 in domain 4.

FIG. 14C depicts mixing ratios along the short route observed and simulated using SGS mixing closure KM5 in domain 4.

FIG. 15A depicts a simulated plume based on weather and CH₄ emission measurements taken along a road (dashed line) at 0600 local time.

FIG. 15B depicts a simulated plume based on weather and CH₄ emission measurements taken along a road (dashed line) at 0900 local time.

FIG. 16 is a process flow diagram for a method of evaluating a fugitive gas emission.

### ABBREVIATIONS

CAMS: Copernicus Atmosphere Monitoring Service,
CH₄: methane,
CONUS: continental United States,
CO: carbon monoxide,
CO₂: carbon dioxide,
°C: degrees Celsius,
EPA: United States Environmental Protection Agency,
FOG: Fuel-Based Oil and Gas,
GFS: Global Forecast System,
GHG: greenhouse gas,
h: hour,
hPa: hectoPaschal, millibar,
kg: kilogram,
km: kilometer,
LDAR: leak detection and repair,
LES: large eddy simulation,
m: meter,
MCF: million cubic feet,
mi: mile,
min: minute,
mph: miles per hour,
NWP: Numerical weather prediction,
%: percent,
ObsPack: Observation package,
OCO-2: Orbiting Carbon Observatory-2,
OGI: optical gas imaging,
OU: University of Oklahoma,
PBL: Planetary boundary layer,
ppb: parts per billion,
ppm: parts per million,
Psi: pounds per square inch,
RRTM: Rapid radiative transfer model,
2°: secondary,
s: second,
SO₂: sulfur dioxide,
SRTM: Shuttle Radar Topography Mission,
TCCON: Total Carbon Column Observing Network,
3°: tertiary,
3D: Three-dimensional,
TKE: turbulence kinetic energy,
TROPOMI: Tropospheric Monitoring Instrument,
Tg: teragram,
T2: temperature at 2 m above the ground,
UAS: Uncrewed Aircraft System,
VPRM: Vegetation Photosynthesis and Respiration Model,
WRF: Weather Research and Forecasting.

### DETAILED DESCRIPTION

The present disclosure is directed to a method which combines a computational atmospheric model with real-world data input and validation to assess the quantity of methane in a methane gas plume as measured by an in situ sensor downwind of the site in various atmospheric conditions. The method is capable of predicting the dispersion plume from a methane super-emitter source within a few meters of wind drift from about a mile away several hours into the future. This allows the user to confirm the concentration of methane emitted from a source and distinguish it from neighboring sources without needing to cross the operator's fence or property line. The plume predicted and observed by the presently disclosed methods closely approximates the plume seen from aerial and spaceborne sensors as the method assesses several pressure-regions of the atmospheric boundary layer. The prediction of plume for fugitive gaseous emissions can be used to identify the source of fugitive gases and to estimate the volume of gases leaked from the source.

Before further describing various embodiments of the present disclosure in more detail by way of exemplary description, examples, and results, it is to be understood that the embodiments of the present disclosure are not limited in structure and application to the details as set forth in the following description. The embodiments of the present disclosure are capable of being practiced or conducted in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the present disclosure has been described in terms of particular embodiments, it will be apparent to those of skill in the art that variations may be applied to the apparatus and/or methods and in the steps or in the sequence of steps of the methods described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

Where used herein, the pronoun “we” is intended to refer to all persons involved in a particular aspect of the investigation disclosed herein and as such may include non-inventor laboratory assistants and collaborators working under the supervision of the inventor(s).

As utilized in accordance with the apparatus, methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the terms “at least one” or “plurality” will be understood to include one as well as any quantity more than one, including but not limited to, 2, 3, 4, 5, 6, 7, 8, 9, 10, 15, 20, 30, 40, 50, 100, or any integer inclusive therein, and/or any range described herein. The terms “at least one” or “plurality” may extend up to 100 or 1000 or more, depending on the term to which it is attached; in addition, the quantities of 100/1000 are not to be considered limiting, as higher limits may also produce satisfactory results. In addition, the use of the term “at least one of x, y and z” will be understood to include x alone, y alone, and z alone, as well as any combination of x, y and z.

Where the specification or claims refer to “an additional” element, that does not preclude there being more than one of the additional element. It is to be understood that where the claims or specification refer to “a” or “an” element, such reference is not to be construed that there is only one of that element. It is to be understood that where the specification states that a component, feature, structure, or characteristic “may,” “might,” “can” or “could” be included, that particular component, feature, structure, or characteristic is not required to be included.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “a, b, c, or combinations thereof” is intended to include at least one of: a, b, c, ab, ac, bc, or abc, and if order is important in a particular context, also ba, ca, cb, cba, bca, acb, bac, or cab. Continuing with this example, expressly included are combinations that contain repeats of one or more item or term, such as bb, aaa, aab, bbc, aaabcccc, cbbaaa, cababb, and so forth. The skilled artisan will understand that typically there is no limit on the number of items or terms in any combination, unless otherwise apparent from the context.

Throughout this application, the terms “about” and “approximately” are used to indicate that a value includes the inherent variation of error for the composition, the method used to administer the composition, or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately”, where used herein when referring to a measurable value such as an amount, a temporal duration, thickness, width, length, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 75% of the time, at least 80% of the time, at least 90% of the time, at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-30 therefore includes 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, as well as sub-ranges within the greater range, e.g., for 1-30, sub-ranges include but are not limited to 1-10, 2-15, 2-25, 3-30, 10-20, and 20-30. Reference to a range of 1-50 therefore includes 1, 2, 3, 4, 5, 6, 7, 8, 9, 10, 11, 12, 13, 14, 15, 16, 17, 18, 19, 20, 21, 22, 23, 24, 25, 26, 27, 28, 29, and 30, etc., up to and including 50. Reference to a series of ranges includes ranges which combine the values of the boundaries of different ranges within the series. Thus, to illustrate reference to a series of ranges, for example, a range of 1-1,000 includes, but is not limited to, 1-10, 2-15, 2-25, 3-30, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 1 mm to 10 m therefore refers to and includes all values or ranges of values, and fractions of the values and integers within said range, including for example, but not limited to, 5 mm to 9 m, 10 mm to 5 m, 10 mm to 7.5 m, 7.5 mm to 8 m, 20 mm to 6 m, 15 mm to 1 m, 31 mm to 800 cm, 50 mm to 500 mm, 4 mm to 2.8 m, and 10 cm to 150 cm, such that any two values within the range of 1 mm to 10 m (or any other range described herein) can be used to set lower and upper boundaries of a range in accordance with the embodiments of the present disclosure.

In addition, techniques, systems, subsystems, and methods described and illustrated in the various embodiments as discrete or separate may be combined or integrated with other systems, modules, techniques, or methods without departing from the scope of the present disclosure. Other items shown or discussed as coupled or directly coupled or communicating with each other may be indirectly coupled or communicating through some interface, device, or intermediate component whether electrically, mechanically, or otherwise. Other examples of changes, substitutions, and alterations are ascertainable by one skilled in the art and could be made without departing from the spirit and scope disclosed herein.

The inventive concepts of the present disclosure will be more readily understood by reference to the following examples and embodiments, which are included merely for purposes of illustration of certain aspects and embodiments thereof, and are not intended to be limitations of the disclosure in any way whatsoever. Those skilled in the art will promptly recognize appropriate variations of the apparatus, compositions, components, procedures and method shown below.

Returning now to the detailed description, the present method and system, in a non-limiting embodiment may use a Weather Research and Forecasting (WRF) model within which is embedded a greenhouse gas (GHG) model. The resulting model (WRF-GHG) has been previously applied for regional CH₄ simulations with a grid spacing only as small as 1-2 km. Thus, it has not been able to resolve individual small-scale plumes emitted from a gas or oil site, which may only spread in a few hundred meters in length and a few tens of meters in width. In the present method, WRF-GHG has been configured and applied at a grid spacing down to 30 m to simulate the plumes from oil and gas production or transportation sites, and to estimate CH₄ emission rate given measured CH₄ enhancement. A 30 m grid spacing is a scale that can resolve the atmospheric turbulent eddies. In pure meteorological research, simulations with such a resolution are called large eddy simulations (LES).

In exemplary embodiments, simulations produced by WRF-GHG in an LES mode were further refined through combination with high-resolution terrain data. In some embodiments, terrain data with a 30 m resolution was obtained from Shuttle Radar Topography Mission (SRTM). The presently disclosed methods can be used to predict where the plume will migrate over time, even under variable or changing weather conditions. This predictive capability is not found in any prior technologies that attempt to characterize methane gas plumes. Additionally, the methods disclosed herein for developing plume simulations are well suited for providing the information necessary to determine the source of fugitive emissions and the quantification of such emissions.

A novel aspect of the presently disclosed method is the use of mobile, terrestrial sensors to validate the quantification of gas plumes predictively modeled with computer resources. The computer portion uses nested simulation by the WRF model with GHG, coupled with the Copernicus Atmosphere Modeling Service (CAMS) Global Near-Real-Time data. The nested WRF-GHG simulations cover 4 layers of domains with individual pixels sizes of 12 km, 800 m, 160 m, and 32 m, respectively. The inner domains zoom in a smaller region gradually with a finer grid spacing. The outer most domain covers the continental United States (CONUS) while the inner most domain covers 3×3 square miles. The outer domain simulation provides the lateral boundary information for the inner domains. The inner most domain has a grid spacing of 32 m which can resolve the atmospheric turbulence. Land elevations were taken from the SRTM data at a resolution of 1 arc-second (30 meters).

For example, this can be envisioned by indicating that a 301×301 grid of these pixels covers the entire state of Oklahoma (750×750 mi), several counties in Oklahoma (200×200 mi), the environs of a city (22×22 mi.), and a few country roads near a city (3×3 mi). Each of these data layers are calculated, meaning that regional-scale data are made available when a site-scale test is run. Multiple subregions may be nested within a single upper-level region, reducing the computational complexity for end users. The LES mode is traditionally not used for simulation of atmospheric constituents since it is computationally expensive, and it has never been proven viable. A field experiment, described below, was undertaken using real plume data to demonstrate that LES CH₄ simulation can correctly capture a small scale CH₄ plume. Coupling methane atmospheric chemistry to the atmospheric boundary conditions worked surprisingly well in LES mode.

By applying this in combination with traditional sensing techniques, the computational predictive model becomes a real-world method capable determining the concentration of methane gas observed at a point within a few hundred meters away from the original source with a reasonable level of accuracy and precision regarding the concentration of the gas and the position of the plume. This positioning is important, because at the spatial scale used in the LES model, a simple driving survey (e.g., equipping a passenger automobile with a fugitive gas sensor and driving it around some county roads) can be used to identify and confirm the concentration predicted by the plume model. This means that an application of this method may be used to distinguish complex plumes from multiple sources in a region.

In the past, emissions simulated with WRF-GHG models have been based on guesses or other field observations e.g., an operator's mass flow loss measurements from online sensors or OGI observations by detection specialists. This is important because most models depend on emissions previously reported and publicly listed in the EPA emissions inventory. However, the EPA inventory is at best incomplete or incorrect, and at worst it is falsely reported in the hopes of reducing emissions-related fines. However, inventory values are important for predicting the background from other high-emissions sources since these values are included in the low boundary conditions. The ability to assess the emission plume based on the non-reported emission is novel when applied to WRF.

The novel methods disclosed herein include, in particular aspects, (1) high spatial resolution of the final model, (2) nested information available through the model, (3) the ability to pull out quantifiable concentrations at a distance, (4) the ability to verify the position of the plume, and (5) the predictive capacity of the model for real-world verification in the future. In a particular embodiment of the method, an investigator uses this technology in both the (1) cyber domains and the (2) physical domains. These ideally interact and operate simultaneously. Below, the stepwise method is separated into these two domains for the purpose of explanation.

In at least one non-limiting embodiment, the modeling of the gas emissions of the present disclosure includes the following steps within cyber domain. First, an investigator installs a WRF-GHG model. A SLURM script for cluster computers may be used to install WRF. Next, the investigator creates a working directory which contains relevant input data. The first investigator next creates the nested domains the investigator wishes to use per standard WRF geogrid methods. The investigator changes the focus of the nested domains to the site of interest as determined by where the corners are located by WRF in terms of the pixel/matrix point in the upper-level domain. A calculator for determining the exact value of this point from more understandable latitude/longitude data is included in the default installation of WRF.

The investigator includes, by copying from an older version in the example script, relevant input data to the model. This includes the native WRF files which describe atmospheric and terrestrial properties (e.g. soil parameters) as well as non-standard imports which are essential to the claims of this project including CAMS Global datasets. It is worth noting that WRF pulls in CONUS meteorology information by default. No action is required by the investigator to obtain CONUS-scale data like wind and temperature estimates. The investigator performs normal WRF steps, which may include constructing the grid for the site, extracting data from the global meteorological data in binary grib format, creating meteorology data for the model domains, and creating the meteorological initial and lateral boundary conditions in WRF-ready format. The process continues with the steps of extracting GHG initial and lateral boundary conditions from global GHG model (i.e., CAMS) outputs using a modified mozbc utility. The mozbc tool is a preprocessor tool used for the WRF-Chem atmospheric modeling system. WRF is run on these domains.

Next, the super-emitter estimation is made on the grid. This adds the estimated emission rate at a point in the domains. Note that this injected value depends on the physical domain information as determined by the steps in the physical domain explanation. The WRF can then be re-run on these domains with the new data. With the data produced, the investigator may either look at the raw output files using a NetCDF4 format file viewer or use other scripts to visualize the data. In a non-limiting embodiment, this method is run every 6 hours in the case of long periods of observation to produce predictions. Longer-term predictions may be less accurate. A computer running 40 modern CPU cores is capable of generating predictions on a previously evaluated domain in less than 2 hours.

In the physical domain, the investigator can take measurements from the field using mobile or stationary fugitive gas detection sensors to validate the models produced in the cyber domain. In some cases, the investigator obtains the emission rate from a suspected leak point of interest. This value may be obtained from several different sub-methods. For example, the investigator can obtain mass flow information from the pipes and equipment related to the leak. By assessing the mass flow upstream from a leak and downstream from the leak and comparing these values, the investigator may obtain mass lost between those points (e.g., a “mass balance” calculation to estimate the rate of loss or the total quantity lost through the leak). If sufficient information about the methane content of the natural gas lost at a well is available, the mass of methane is calculated. The mass leak rate does not precisely correspond to the volumetric or concentration leak rate, but this difference is below the sensitivity of the method in practical examples. The value is passed to the super-emitter estimation in the cyber domain. Additionally, or alternatively, the mass or volumetric flow rate may be observed with sensors external to the leak. In one example, a UAS drone with an OGI camera capable of quantification estimation to determine the leak rate. The value is passed to the super-emitter estimation in the cyber domain.

In some embodiments, the investigator uses an in situ sensor (mobile or stationary instruments) with sufficient concentration limit-of-quantification, sensitivity, selectivity, and precision to observe minute fluctuations in atmospheric methane concentrations (sub-ppb concentration sensitivity) to investigate the real-world concentration at individual point(s). When the sensor is co-located with the predicted plume from the emission of interest, the investigator records the timing (and possibly position) of the detection event. The investigator compares the observation at that point with the prediction made by the model. If the values are similar, the plume prediction is determined to match the observation. Ratios between the observed and simulated plume concentrations can be multiplied on the prescribed emission rate to estimate the real released emission rate.

As previously noted, this disclosure describes a novel method for detecting and quantifying the diffusion of methane released in a methane gas plume. In the present work, a field experiment at a site in Pampa, Texas (the “Pampa site” or “release site”) was conducted using two CH₄ sensors to detect controlled CH₄ releasees. Suitable gas sensors include the LI-COR model 7810 trace gas analyzer and OGI cameras, which are available from a number of commercial sources, including Sierra-Olympia Technologies, Inc.

The experiments disclosed herein were focused on the investigation of whether high-resolution simulations using the WRF-GHG model could accurately simulate the released CH₄ plumes in the presence of the evolving boundary layer structure from sunrise to noon time (12:00 PM). The WRF-GHG model was applied with a very high resolution with a grid spacing of tens meters. Thus, the model is essentially applied in an LES mode. The in situ ground-truth measurements from the experiment provide an opportunity for evaluation of the CH₄ LESs and such LESs are used to examine the dynamics of CH₄ plumes in presence of the transition of atmospheric boundary layer during the day.

In prior work, the WRF-VPRM, a weather-biosphere-online-coupled model in which the biogenic CO₂ fluxes are handled by the Vegetation Photosynthesis and Respiration Model (VPRM), was improved and applied to examine CO₂ fluxes and concentrations at regional scale. In the present experimental, an enhanced version of WRF-VPRM by including CH₄ (referred to as WRF-GHG hereafter) was further developed by coupling with the CAMS CH₄ global simulation for the initial and boundary conditions and ingesting the WetCHARTs wetland CH₄ emissions and anthropogenic CH₄ emissions. WRF-GHG has been shown to capture the monthly variation of concentrations and episodic variations of CO₂ and CH₄ associated with frontal passages over United States, evaluated using multiple data sets, including remotely sensed data from the TCCON, OCO-2, TROPOMI, and in-situ measurements from the GLOBALVIEW ObsPack data.

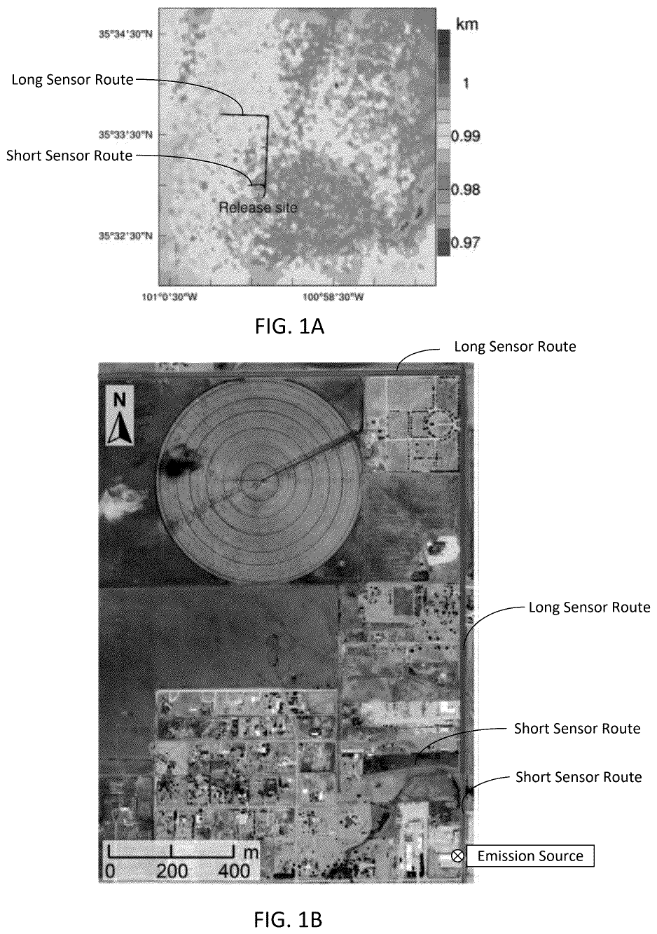

To guide the field experiment at the Pampa site and to examine the impact of meteorology on CH₄ plumes during the field experiment, a quadruply-nested WRF-GHG domain was set up with sequentially refined grid spacing, i.e., 12 km to 0.8 km to 0.16 km and to 32 m. The outermost domain covers the contiguous United States, the second domain covers the Texas panhandle region, while the third and the fourth domain zoom-in on the Pampa site. Only the first two domains were activated for real-time forecasting to guide the field experiment, and all 4 domains were activated for detailed retrospective LES of CH4. Land elevations were taken from the SRTM data at a resolution of 1 arc-second (30 meters). FIG. 1A shows the land elevations in the innermost domain. Two land use datasets were used: the MODIS land use with a 1 km resolution and the National Land Cover Database (NLCD) with a 30 m resolution. The higher-resolution NLCD land use leads to a smoother temperature field in the early morning, while its impact on flow dynamics and CH4 plume behavior at the research site in the presence of southerly winds is minimal (figure not shown). Therefore, only the NLCD results are presented in the following sections. The model domain has 47 vertical layers extending from the surface to 10 hPa with 21 layers in the lower 2 km above the ground to resolve boundary layer processes.

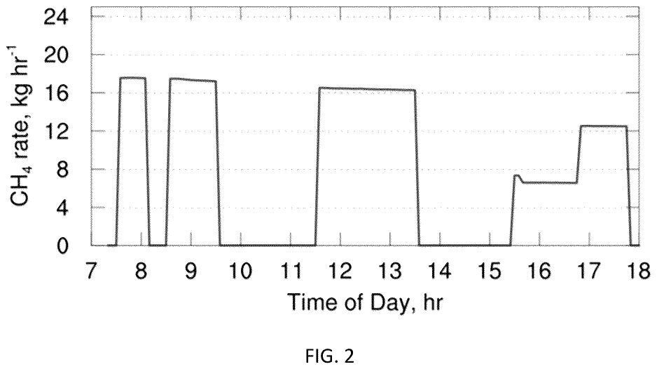

The operational Global Forecast System 234 (GFS) 0.25-degree-resolution data provided meteorological initial and boundary conditions and the CAMS 3°×2° global inversion-optimized concentrations provided initial and boundary conditions for CH4. Anthropogenic CH4 emissions were taken from the 4 km×4 km Fuel-based Oil and Gas (FOG) Inventory. The CAMS initial and boundary CH4 conditions and the coarse FOG emissions provided the background CH4 concentration. A CH4 emission rate of 17.5 kg hr-1 was added at the Pampa site based on the CH4 controlled release rate, which varied by only 2% on the morning of the field experiment day. CH4 plume from this point source is the focus of this study. While the CH4 emission rate is applied through the whole simulation period, the actual controlled release occurred randomly (FIG. 2).

Selected major physics parameterization schemes within WRF-GHG include the Dudhia shortwave radiation scheme, the rapid radiative transfer model (RRTM) for longwave radiation, the Noah land surface model, and the Shin-Hong planetary boundary layer (PBL) scheme. Selection of PBL schemes is critical for accurate simulation of lower tropospheric CH₄ vertical distribution. The Shin-Hong scheme is a scale-aware nonlocal scheme with explicit treatment of entrainment fluxes, which has been shown to be critical to reproducing boundary layer structures. The Shin-Hong scheme adopts a nonlocal heat flux profile fitted to LES results, and achieves scale-awareness (or horizontal grid spacing dependency) through scaling both local and nonlocal eddy fluxes according to the normalized grid spacing. Since the third and fourth domains reach LES resolutions, PBL scheme was turned off and a 1.5 order three-dimensional turbulence kinetic energy (TKE) closure diffusion scheme was used for sub-grid vertical mixing, following previous one-way nested WRF-LES studies. Two additional sensitivity simulations were also conducted using the Smagorinsky first order closure and Scale-aware 3D TKE LES/PBL scheme for sub-grid vertical mixing. Since no precipitation occurred during the experiment, cumulus and microphysics schemes were deemed less relevant.

The WRF-GHG real-time forecasting with only the outer two domains was launched daily before the experiment. These forecasts were used to select a suitable day for measuring the CH4 plume on the north side of the Pampa site. Following the field experiment on the testing date, nested WRF-GHG LES runs with full 4-layer domains were conducted to examine CH₄ plumes within a changing boundary layer through the day. The fine horizontal and vertical resolutions used in this study can better resolve small-scale processes and shallow surface layer, which is critical for accurately reproducing the formation of the CH₄ plumes within a few hundred meters or kilometers under different conditions.

The field experiment is based on the controlled CH₄ release at a testing site at the Pampa facility (the Pampa site), which inherently depends on the site availability, wind conditions, and other site-specific constraints. The WRF-GHG real-time forecasting was monitored daily to ensure the optimal weather and plume conditions. On the testing date, the Pampa site performed controlled releases of natural gas to enable the evaluation of sensor technologies and WRF-GHG LES. Meanwhile a suite of instrumentation was deployed to measure the released CH₄ plumes. The emission site had a large southerly wind fetch with minor obstructions to other directions (see satellite image in FIG. 1B). Instrument location and co-locations were determined during this field study for southerly wind conditions. Land access permissions were obtained from landowners to the north of the emission site prior to the experiment. A natural gas leak was simulated by using a mass-flow metered release of natural gas from the Pampa, TX city supply. The CH₄ volumetric concentration of the natural gas source was reported as 87.56% by online gas chromatography at the release point. The maximum expected volumetric flow rate of gas from the emission source was 26.0 MCF/day. Experiment participants were given a randomized release schedule which varied the release pressure up to 15 psi from a 2-inch orifice, corresponding to a 30% change of the maximum flow rate. Gas was released in a single blind design, which meant that researchers operating sensors on routes did not know either the exact release timing or the pressure of the release.

The field work was supported by a host of instruments, including a LI-COR 7810 i situ trace gas analyzer. This instrument was fitted with an inlet tube and filter. The inlet tube was placed outside the window of a Honda Odyssey van, just above the roofline, on the port side of the vehicle to sample gas acquired during motion from the slipstream of the vehicle. The vehicle was driven <10 mph, where possible, to produce regular sampling. The vehicle was driven on areas with explicit landowner permission and public right-of-ways only. The short and long routes are shown in FIGS. 1A and 1B. The field work was also supported by a Sierra Olympic Ventus OGI camera mounted on a DJI Matrice 300 Uncrewed Aircraft System (UAS) and a handheld camera used to visualize CH₄ plumes. The camera was operated by site personnel during the release to visualize plumes of gas emitted from the emission source. These observations confirmed that the sensors e.g., the LI-COR was reporting emission enhancements due to gas produced by this leak. Videos from these flights were recorded by the site personnel. Videos were analyzed for gas concentration using Teledyne's quantitative OGI interface. Similar analysis technologies are reported to the literature with a median relative error of 24.4% at emission rates above 1 kg·hr⁻¹. The OGI camera was operated by the anonymous industry partners.

Meteorology at the release site was monitored using a Lufft WS500-UMB all-in-one meteorology sensor connected with a Campbell-Scientific CR6 logger. These instruments recorded 1-min samples and 5-min rolling average data. The all-in-one meteorology sensor was mounted 5 m above ground level. Pressure was monitored at ground level using a Vaisala PTB 330 sensor incorporated with the same datalogger. The meteorology sensors were operated by the OU team.

During the field work, natural gas was released following a single-blind schedule such that the site operators knew the timing and flow rates of the release, but the OU measurement team did not. The measurement team anecdotally noted sudden changes in observed concentrations (e.g., alternating periods of apparent CH₄ enhancement and those with no detected enhancement), which aligned with changes in release rate revealed after the experiment. This indicates that the LI-COR sensor produced reasonable and reliable data quality during the experiment. For example, between 8:10 and 8:30 AM local time, no CH₄ enhancements were observed, which coincided with a pause in the controlled release (FIG. 2).

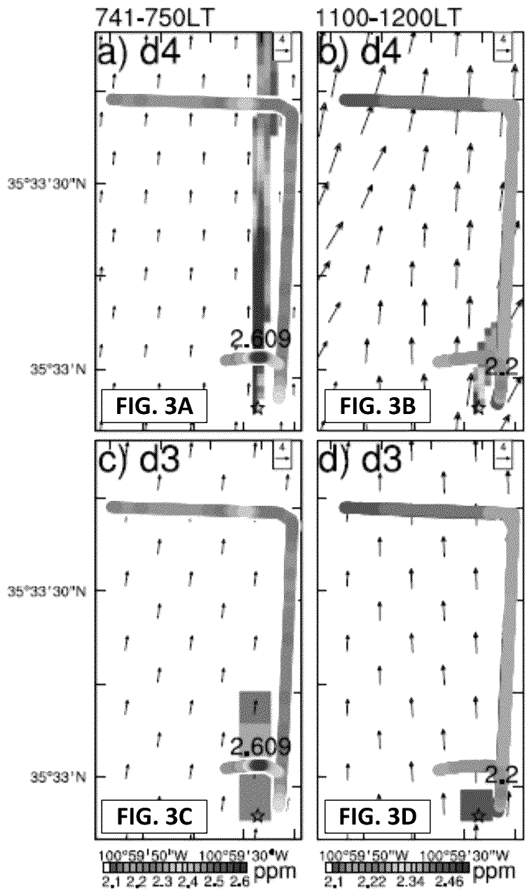

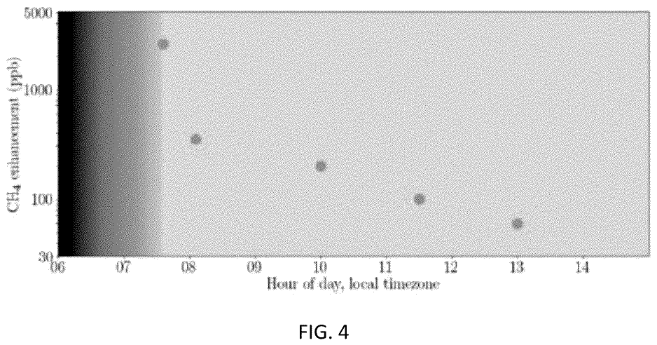

The observed CH4 enhancement varied significantly throughout the day. Note that the CH4 plumes exhibited bell-shaped patterns, with the CH4 enhancement defined as the increase in concentration above the background level at the edges of the bell shape. The enhancement at the short route (250 m north of the release site) decreased through the experiment from early morning to afternoon (FIGS. 3 & 4). On March 28, the sunrise time in Pampa was 7:36 AM. At sunrise, a peak concentration of 4.8 ppm, i.e., an enhancement of 2.6 ppm over the background 2.2 ppm, was observed during a transect from 7:36 to 7:41 AM, and an enhancement of 0.4 ppm during 7:41-7:50 AM (FIG. 3(a) & 4), corresponding to the coldest time of the day (FIG. 5(a)) was observed. The enhancement at the short route decreased to 100 ppb through the course of the morning (FIG. 4) and fell below 100 ppb between 11:00 AM and 12:00 PM (FIG. 3(b)). After 12:30 PM, the enhancement dropped to below 60 ppb. All these enhancements were recorded along the short route (~250 m from the release point). At the long route (1.5 km from the release location), no significant enhancements were detected, except a 100 ppb enhancement at 7:45 AM (section in light yellow on the northernmost leg of the long route in FIG. 3(a)). Since the released plumes experienced the most substantial changes from sunrise to noon (FIG. 4), the subsequent analyses mainly focuses on the CH4 variation and the influential factors during this period.

The controlled CH4 release before 1:30 PM maintained roughly the same rate when the release was turned on (FIG. 2). CH4 emission rate ranged between 17.56 and 17.19 kg hr−1 (only by 2%) during 7:35-9:30 AM (except during a brief pause from 8:10 to 8:30 AM). Thus, the significant variation in CH4 concentrations (by two orders of magnitude, from ppm to tens of ppb) during each of the observation trips must have been dictated by atmospheric variability. The WRF-GHG LES in domain 4 with a grid spacing of 32 m generally reproduced the atmospheric flow characteristics of the site and the CH4 plumes. Simulated CH4 concentration at the ground level aligned well the concentrations and narrow plume widths (60-90 m) of the CH4 plumes as observed by the LI-COR sensor from sunrise to noon (FIG. 3(a-b)). This indicates the WRF-GHG model correctly captured the impact of boundary layer meteorology on CH4 plumes as the ABL evolved throughout the day. In comparison, in domain 3 with a grid spacing of 160 m, the model could not resolve the measured plume at such close proximity and underestimated the plume enhancement by ~400 ppb at 7:50 AM (FIG. 3(c)). In the grid cell containing the release point, the emission was diluted 25 times in a 160 m grid cell compared to a 32 m grid cell in domain 4, leading to this substantial underestimation. Thus, the results of the WRF-GHG simulation in domain 4 are highlighted in the review of this study.

The measured surface temperature at the Pampa site, winds at the MADIS site in Pampa, and the balloon sounding in Amarillo, TX (75 km to the southwest of the Pampa, TX) were used to evaluate the lower tropospheric meteorology simulated by WRF-GHG (FIGS. 5 & 6). WRF-GHG reproduced the observed surface temperature quite well in domain 4 (FIG. 5) with a mean bias of −0.3 °C (NMB of −2.4%) and underestimated surface wind speed by −17.1%. This performance is comparable to operational NWP simulations. The WRF-GHG simulation started integration at 7:00 AM. After a half-hour spinup, the simulated surface temperature aligned well with observations after 7:30 AM. However, the simulated temperature showed more variation than observations after 11:30 AM, likely due to the impact of large turbulent eddies (as will be discussed later). Because WRF-GHG was run in LES mode with high resolution (a grid spacing of 32 m), the simulation was capable of resolving large turbulent eddies that are ubiquitous and random in the daytime convective boundary layer. These large eddies induce temperature variation. The large air temperature variation and wind vector variation caused by individual large eddies were well resolved by the model (manifested in the instantaneous fields simulated by WRF-GHG) after 11:30 AM (FIG. 5). In comparison, the observations were 5-min averages from the Lufft WS500-UMB meteorology sensor, so the variations due to large eddies were likely smoothed out. Thus, the time series of observed temperature appeared smoother than that of the simulated temperature.

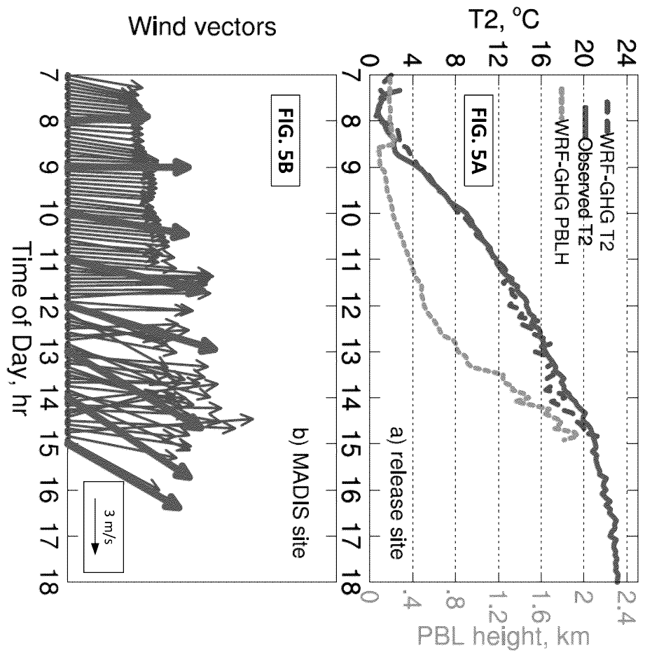

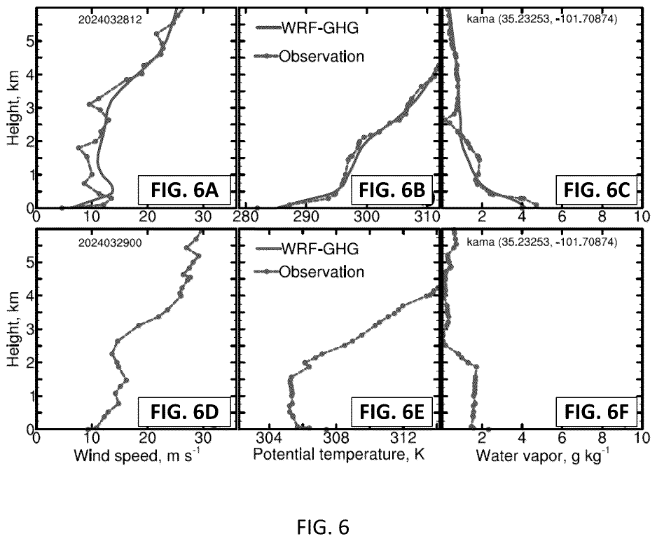

In addition to the surface temperature, the atmospheric boundary structure/stability was examined in FIG. 6. Potential temperature (θ) profiles provide a straightforward and meaningful representation of stability, with inversions representing stable conditions and decrease with height indicating unstable conditions. The simulated and observed profiles of wind speed, potential temperature, and water vapor mixing ratios in Amarillo, TX show an excellent agreement at 7:00 AM, suggesting a very stable shallow boundary layer (~200 m) where water vapor was confined (FIG. 6 (a, b, c)). Twelve hours later at 7:00 PM, a well-developed convective boundary layer was observed as shown by the balloon sounding, with the capping inversion (indicating boundary layer top) residing at ~2 km above the ground (FIG. 6 (d, e, f)). Consistent with observations, the simulated convective boundary layer height varies from 100 m in the early morning to ~2 km in the afternoon (FIG. 5(a)). Large turbulent eddies normally have scales resembling the boundary layer height. Thus, large eddies on this day should have scales of 1-2 km, as confirmed by WRF-GHG LES later. Note that routine operational soundings were available only twice a day.

The simulated boundary layer height increased over time except during a transition period around 8:30 AM from nighttime stable boundary layer to daytime convective boundary layer, when the boundary layer height appeared to collapse. This apparent collapse was deceiving and it was actually due to the different treatment of stable and convective boundary layers in the Shin-Hong PBL scheme and its predecessor, Yonsei University (YSU) scheme. In both schemes, a critical Richardson number of 0.25 is used to diagnose boundary layer height under stable conditions; while for unstable condition, a critical Richardson number of 0 is used. Thus the simulated “collapse” in the early morning transition actually indicates boundary layer growth from stable to convective boundary layer.

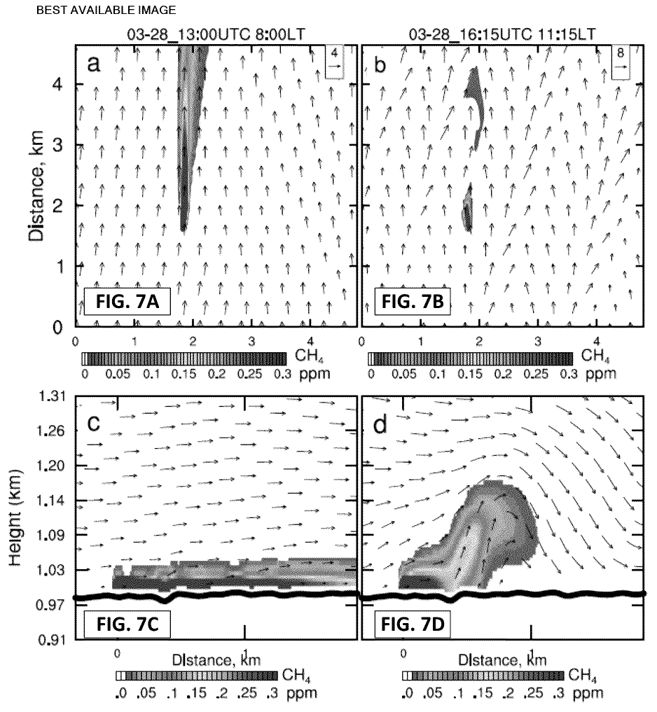

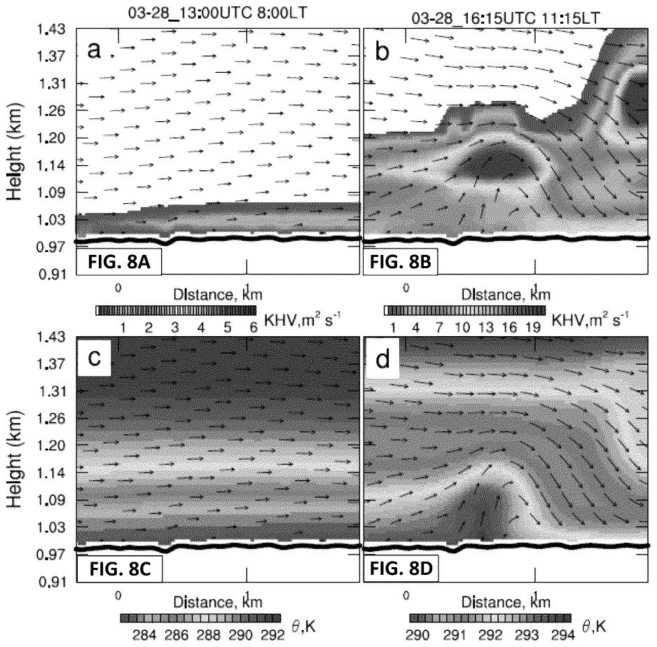

These boundary layer and turbulence variations dictate the dynamics of CH4 plumes. Horizontal distributions and vertical cross-sections of simulated CH4 plumes at 8 AM and 11:15 AM are compared in FIG. 7(a-d), which are representative of early morning stable boundary layer and daytime convective boundary layer. During the early morning, the CH4 plume was confined within a shallow layer near the surface and lengthily stretched downwind. There was an enhancement of about 100 ppb beyond 1.6 km (1 mi) from the release site. The CH4 plume was barely dispersed vertically, limited by the high stability during the early morning as shown in the observed and simulated profiles (FIG. 6) and the vertical cross-section of potential temperature (FIG. 8(c)). Potential temperature shows a compact layered structure, indicating a very stable boundary layer and thus a very small vertical mixing coefficient (<1 m² s⁻¹, FIG. 8(a)).

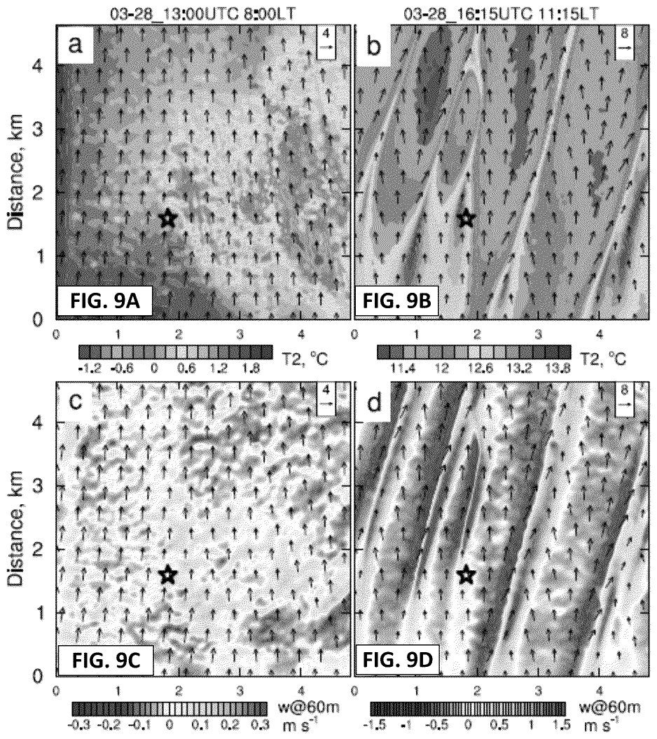

When approaching local noon, the surface CH4 plume became shorter and was transported upwards about 300 m from the release site, driven by large turbulent eddies in the convective boundary layer. These large eddies can be clearly seen from the spatial distribution of temperature and vertical velocity (FIG. 9(a-d)). During the early morning, the temperature distribution exhibited a patchy pattern, reflecting the initial patchy distribution of land properties with a grid spacing of ~1 km in the MODIS land use. This indicates that when turbulent eddies were not yet developed in the early morning, the surface temperature was primarily determined by the surface energy balance, which was dictated by land surface properties including land cover and soil properties. Using the higher-resolution NLCD land use, the patchy pattern disappeared (FIG. 9(a)). Later at 11:15 AM, the spatial distribution of temperature and winds was dominated by large eddies approximately 1 km wide in the west-east direction. Such a scale is comparable to the boundary layer height indicated by the afternoon sounding on this day (FIG. 6). As the turbulent eddies actively developed, the spatial distribution of surface temperature and winds became less tied to land surface heterogeneity. Unlike the cellular structures under weak wind conditions, the large eddies encountered during the experiment manifest as elongated roll vortices likely due to strong surface winds (~10 m s⁻¹, FIG. 6 (a, d)). The large eddies and flow streamlines follow potential temperature contours or isentropes as shown in vertical cross-sections (FIG. 8(d)) given that potential temperature is conserved along air parcel trajectories when diabatic heating is negligible.

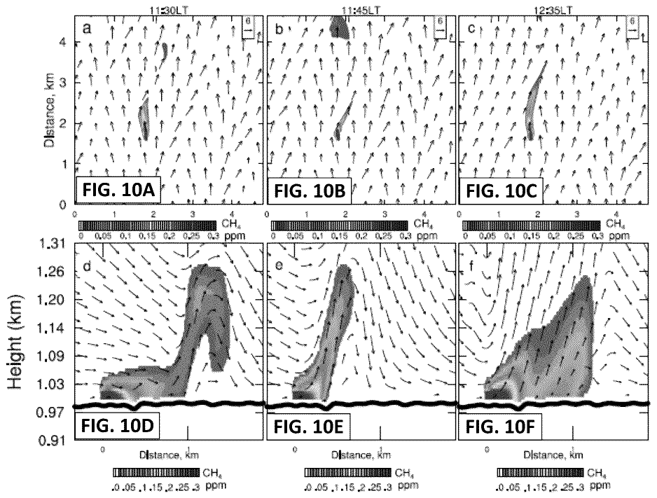

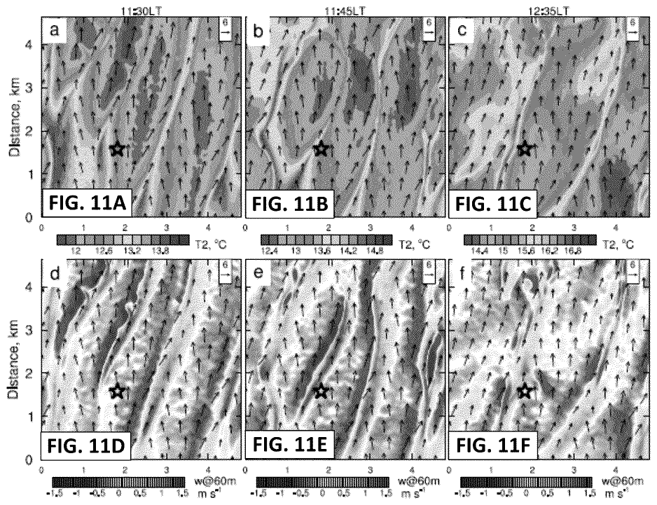

Large eddies cause meandering of the CH4 plumes in the mature daytime convective boundary layer (FIG. 10 (a-f)). The CH4 plume may be captured by a large eddy (as illustrated by spatial distributions of temperature and winds in FIG. 11) at random distances from the release—for example, at 1 km at 11:30 AM, 200 m at 11:45 AM, or immediately from the release along an elongated convergency line at 12:35 PM (FIG. 10). In addition, as indicated by the mixing coefficient (FIG. 8(a, b)), SGS turbulence mixing was larger in the convective boundary layer around noon than early morning. Thus, the surface CH4 plume was quickly dispersed, with CH4 enhancement barely exceeding 100 ppb beyond 300 m downwind the release point (FIG. 10). Such active eddies and their efficient transport of CH4 plume persisted into the afternoon.

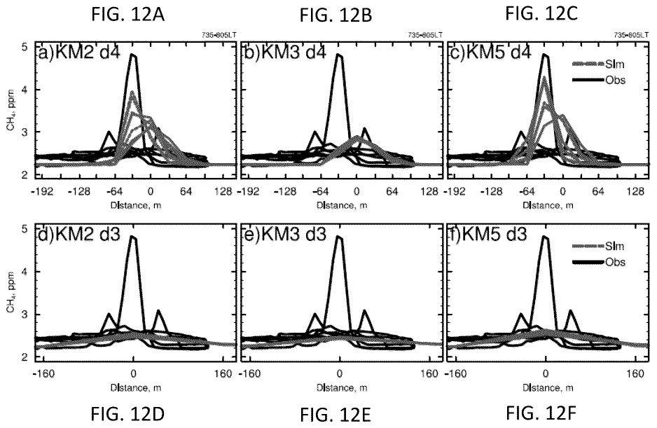

The ratios of simulated to observed cross-plume enhancements can be used to calibrate the greenhouse gas emission rates. Thus, observed and simulated CH4 enhancements along the short route at 250 m from the release during two morning release windows are examined in FIGS. 12-14. The simulated CH4 enhancements at 5-min resolution were more gradual than observations, regardless of which SGS turbulence closure is chosen. The range of simulated CH4 enhancement (0.8-1.8 ppm) was narrower than the observed range (~0.4-2.6 ppm) during sunrise (FIG. 12). The WRF-GHG LES underestimates the enhancement range due to insufficient grid resolution to resolve fine-scale processes and the averaging effect of mixing parameterization. During stable conditions at this time, ubiquitous turbulent eddies have not yet developed, and the near-surface stable boundary layer was characterized by intermittent turbulent events and submeso motions. The WRF-GHG LES model simulates mixing processes through the subgrid mixing parameterization solely and therefore cannot fully resolve these processes. The subgrid mixing parameterization tries to simulate the mean effects of unresolved motions/mixing in the form of an ensemble average. This averaging effect causes WRF-GHG LES to underestimate the variation of CH4 plume impacted by the intermittent smaller-scale motions/mixing. Given the instantaneous and averaging characteristics of observed and simulated CH4 plumes respectively, certain averaging processes for the observed plumes are necessary before using the ratio of simulated and observed cross-plume enhancement to calibrate/scale CH4 emission rates.

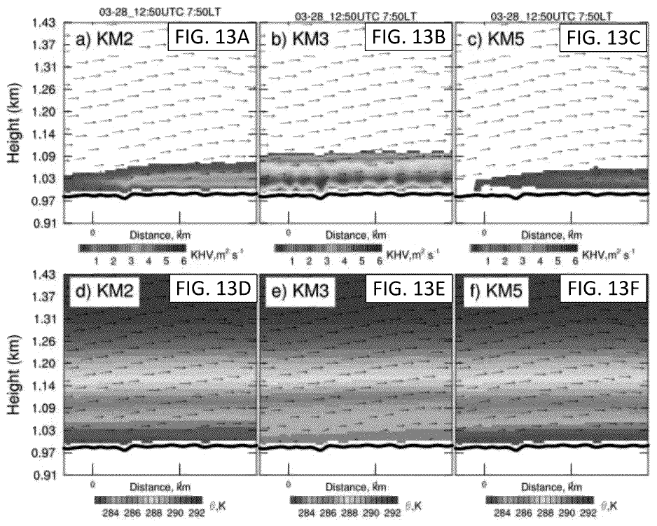

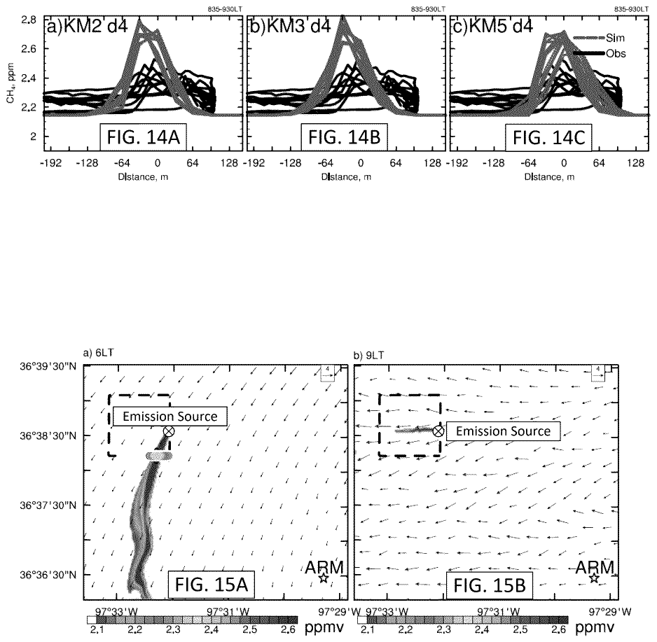

The simulated CH4 plume enhancement is significantly impacted by the grid resolution and different SGS turbulence closures. The simulated cross-plume CH4 enhancements in domain 3 (160 m grid spacing) and domain 4 (32 m grid spacing) are compared in FIG. 12. With a grid spacing of 160 m, the CH4 enhancement is greatly reduced in domain 3 compared to domain 4. As a result, domain 3 is not able to capture CH4 enhancements above 0.5 ppm at sunrise time. Three different SGS turbulence closures were compared. The Smagorinsky closure (KM3) simulates stronger vertical mixing coefficient during the early morning at 7:35-8:05 AM, which reduces the near surface potential temperature inversion compared to that simulated by the other two SGS closures (FIG. 13). As a result, the KM3 simulates more plume dispersion and weaker CH4 enhancement compared to the other two (FIG. 12(a-c)). During the same period, the TKE-1.5 closure (KM2) and the 3DTKE scheme (KM5) simulated CH4 plumes with substantial variations, while KM3 simulated a roughly constant plume (FIG. 12) again due to excessive mixing (FIG. 13). In comparison, during 8:35-9:30 AM, all three closures overestimated the CH4 enhancement by up to 300 ppb (FIG. 14) and the difference between mixing coefficients from the three SGS closures becomes less prominent.

Consistent minor spikes in CH4 concentration were observed originating roughly above a marked high-pressure pipeline along the long route, which appears as a slight enhancement region on the northernmost leg of the long route in FIG. 3 near 100°59′30″ . Such a leak would normally be below the actionable threshold for operators, since the excursion was less than 10 ppb above the roughly 2.2 ppm site background. Few sensors would have the quantification precision necessary to even notice it. It is worth noting that the field site, like other sites in the oil & gas extraction regions, may be subject to similar minor leaks.

While the LI-COR sensor was measuring CH4 on the routes, the OGI camera was deployed on a UAS to visualize CH4 plume at ~10 m above the release site. However, during sunrise on the release date, it was cold (~1 °C). With the sun still too low to sufficiently warm the ground, pipe, and surrounding structures, the temperatures across the entire area were nearly uniform. As a result, the OGI camera could not confidently distinguish the plume, and the quantitative OGI interface was not able to analyze the gas concentration. No video was recorded by the OGI camera later in the day due to high winds (>8 m s−1, FIG. 6(d)), which might cause instability or even damage to the camera. Thus, these conditions represent the limitations of using the OGI camera to detect CH4 plumes. On the afternoon of the previous day with weaker winds, the OGI camera was able to identify the CH4 plume within 20-30 meters of a release at a similar rate (~17 kg hr⁻¹).

The investigation of CH4 plumes in Pampa demonstrated that combining mobile LI-COR 7810 measurements with high-resolution WRF-GHG LES can be used to understand CH4 plume behavior under variable atmospheric conditions, providing guidance for quantifying CH4 emissions from point sources. To further demonstrate its generalizability, a similar approach was used to examine CH4 plumes during the morning transition at a second site, a cattle farm depicted in FIG. 15, which was located ~6 km northwest of the SGP ARM site. The WRF-GHG LES simulation used a prior emission rate of 100 kg hr⁻¹ at the cattle farm. Based on the ratio of observed to simulated CH4 plumes, the farm's emission rate was estimated to be ~65 kg hr⁻¹ at 6:00 AM. Similar to the Pampa experiment, the boundary layer transition during the early morning exerted significant impact on CH4 plumes (FIG. 15). At 6:00 AM, the CH4 plume extended a few kilometers downstream within the stable boundary layer, which explains the nighttime CH4 peaks often observed at the ARM site in the presence of northwesterly winds. At 9:00 AM, the convective boundary layer had already grown, as indicated by the staggered wind vectors induced by large eddies, which led to a shorter surface CH4 plume. At this time, the plume enhancement decreased to tens of ppb beyond 1 km with an emission rate of 100 kg hr⁻¹. The growth of the convective boundary layer occurred earlier (~9:00 AM) on this summer day at the cattle farm compared to the Pampa site (~11:00 AM).

Thus, as noted above, a controlled CH₄ release experiment was conducted at a test site in Pampa, Texas. While an OGI camera was mounted on a UAS to monitor the CH₄ plume directly above the release point, a LI-COR 7810 was deployed on a van to measure downstream plumes along the routes about 250 m to 1.5 km from the release point. Substantial changes in CH₄ plumes were observed throughout the sunrise time. At a release rate of 17.5 kg·hr⁻¹, the maximum CH₄ enhancement measured by LI-COR 7810 was 2.6 ppm at sunrise (7:36 AM), 250 m from the release location. Within half an hour after sunrise, the enhancement dropped to 0.3-0.4 ppm. The enhancement further decreased to 0.2 ppm by 10 AM and was less than 0.1 ppm after 11:30 AM. After 1 PM, the plume enhancement was lower than 60 ppb. At 1.5 km from the release location, a notable CH₄ enhancement of about 100 ppb was observed only at sunrise. Due to the low temperature (1 °C) at sunrise and insufficient thermal contrast, the OGI camera failed to detect the CH₄ plume. Later in the morning, the OGI camera could not operate due to strong winds (>8 m/s). This suggests that the detection and quantification of CH₄ emissions a few hundred meters from a super emitter (>10 kg·hr⁻¹) are possible with appropriate tools (e.g., LI-COR 7810), while the application of OGI camera is limited by weather conditions.

The WRF-GHG LES with a 32 m grid spacing can successfully reproduce the observed CH₄ enhancements. The simulated meteorology was examined in terms of the boundary layer variation and its impact on CH₄ plumes. At sunrise, the near surface air was very stable and the CH₄ plume was barely vertically dispersed. Thus, the CH₄ plume was confined in the very shallow surface layer and lengthily stretched downwind. The model underestimated the CH₄ enhancement at sunrise, likely due to the ensemble average characteristic of the mixing parameterization, which smooths out the intermittent turbulence and its impact on CH₄ plume. After 11 AM, active large eddies in the convective boundary layer effectively dilute the CH₄ plume. Thus, the CH₄ enhancement rarely exceeded 100 ppb beyond 300 m from the release point. The simulated CH₄ plume enhancement is significantly impacted by the grid resolution and different subgrid turbulence mixing parameterizations. A grid spacing of 160 m is inadequate for resolving narrow CH₄ plumes released from the single site. These results provide valuable guidance regarding the detection and quantification of CH₄ emissions, for example, the necessary accuracy and detection range needed. Giving the instantaneous and averaging characteristics of observed and simulated CH₄ plumes respectively, certain averaging processes for the observed plumes are necessary before using the ratio of simulated to observed cross-plume enhancements to calibrate/scale CH₄ emission rates during the early morning.

In summary, accurate identification of CH₄ sources for preventing/reducing CH₄ emissions is often hampered by the inadequate accuracy and spatiotemporal coverage of CH₄ detection, and the lack of accurate CH₄ forward modelling used in top-down inversion systems. This disclosure describes in detail the results of a field experiment conducted using two CH₄ sensors (LI-COR 7810 and OGI camera) to detect controlled CH₄ releases. High-resolution simulations using the WRF-GHG model were examined to determine if these models could accurately simulate the released CH₄ plumes in the presence of the evolving boundary layer structure from sunrise to noon. CH₄ plumes showed a substantial change through the sunrise time. At a release rate of ~17.5 kg·hr⁻¹, the maximum enhancement of CH₄ measured by LI-COR was 2.6 ppm at sunrise time (7:36 AM), 250 m from the release location. Within half an hour after sunrise, this enhancement decreased to 0.3-0.4 ppm. The enhancement further decreased to 0.2 ppm by 10 AM and <0.1 ppm after 11:30 AM. Due to the low temperatures at sunrise, the OGI camera failed to detect the CH₄ plume. Despite the changing boundary layer conditions, the WRF-GHG LES with a 32 m grid spacing successfully reproduced these CH₄ enhancements. In situ measurements in combination with numerical simulations illustrate the impact of the transition from a stable boundary layer in the early morning to a convective boundary layer at noon on the dynamics of CH₄ plumes.

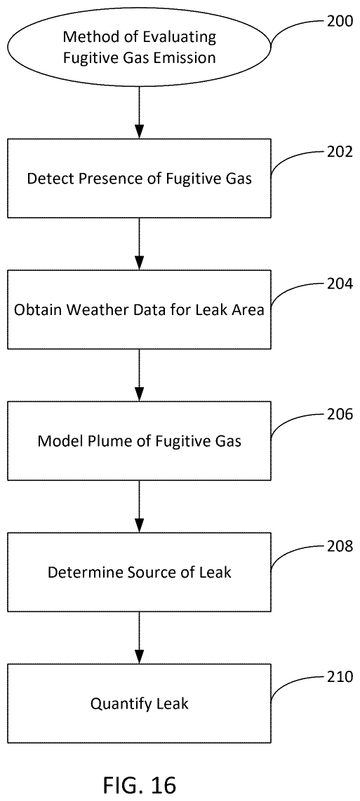

Turning to FIG. 16, shown therein is a method 200 for evaluating a fugitive gas emission. The method 200 begins at step 202 when a fugitive gas is detected or suspected. The fugitive gas can be CH₄, CO₂, SO₂, CO, or another gas that can be accurately detected with field sensors or other equipment. Suitable gas detection equipment includes the LI-COR 7810 and OGI cameras described above. At step 204, weather information is obtained for the area of interest surrounding the detected fugitive gas. The weather information can be obtained from local instruments or from nearby weather monitoring stations. At step 206, a computer-implemented model is deployed to estimate the size and concentration of the gas plume based on the detected concentrations of the fugitive gas and the observed weather conditions. In exemplary embodiments, this step is carried out using the WRF-GHG simulation in a LES mode, with resolution of less than 1 km, less than 500 m, less than 100 m, or less than 50 m. In some embodiments, the grid spacing is selected to be in a range of from about 5 m to about 50 m, including, but not limited to about 10m, about 15 m, about 20 m, about 25 m, about 30 m, about 35 m, about 40 m, and about 45 m. Next, at steps 208-210, the computer model for the gas plume is examined. The model plume can be examined to determine the likely source of the fugitive emission at step 208 (e.g., a super emitter pipeline, wellhead, tank battery, farm, bog or other gas source) or the quantity of gas emitted over time through the plume at step 210. It will be appreciated that the steps outlined in the method 200 of FIG. 16 can be reordered. In some embodiments, not all of the steps identified in FIG. 16 will be implemented by the method 200.

While the present disclosure has been described herein in connection with certain embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus the embodiments described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of methods and procedures as well as of the principles and conceptual aspects of the present disclosure. Changes may be made in the apparatus, components, systems, and methods described herein, or in the steps or the sequence of steps of the methods described herein, without departing from the spirit and scope of the present disclosure. Further, while various embodiments of the present disclosure have been described in exemplary claims herein below, it is not intended that the present disclosure or a future patent application be limited to these particular exemplary claims.

What is claimed is:

1. A method of evaluating a methane gas plume, comprising:
obtaining sensed data by:
using gas sensors at a plurality of gas sampling points to obtain physical observations of methane gas concentration in the methane gas plume, wherein the methane gas plume has an emission source; and
using weather sensors at a plurality of weather sampling points to obtain physical observations of meteorological conditions;
obtaining one or more computer simulations of distributions of the methane gas plume within the atmospheric boundary layer, wherein the one or more computer simulations are operated over multiple nested spatial domains to obtain results of estimated methane gas quantities at one or more locations remote from the emission source; and
combining the sensed data and the results obtained from the one or more computer simulations to obtain quantified estimations of methane gas on a predetermined spatial scale and resolution for the one or more locations remote from the emission source, thereby providing a map of estimated methane gas concentrations of the methane gas plume, wherein the map extends a distance from the emission source of the methane gas plume, and wherein the map comprises a coverage area between the emission source and the gas sampling and weather sampling points.

2. The method of claim 1, wherein the coverage area of the map extends beyond the gas sampling and weather sampling points.

3. The method of claim 1, wherein the map comprises a grid spacing in a range of 5 meters to less than 1 kilometer.

4. The method of claim 1, wherein the map comprises a grid spacing in a range of 5 meters to about 50 meters.

5. The method of claim 1, wherein the physical observations of methane gas concentration in the methane gas plume are obtained by using at least one of (a) two or more stationary in situ methane gas sensors, (b) two or more mobile in situ methane gas sensors, (c) at least one stationary in situ methane gas sensor and at least one mobile in situ methane gas sensor, (d) one or more vehicularly-mounted in situ methane gas sensors, (e) one or more stationary remote sensors configured to sample the lower atmospheric boundary layer, and (f) one or more methane gas concentration measurement devices or schema configured with an integrable spatial component.

6. The method of claim 5, wherein the one or more vehicularly-mounted in situ gas sensors are mounted on at least one of an automobile, a truck, a utility vehicle, an airplane, a boat, and an uncrewed aerial vehicle.

7. The method of claim 1, wherein the one or more computer simulations of methane gas plume distributions are selected from (a) atmospheric meteorology simulations and estimations, (b) terrestrial latent heat simulations and estimations, and (c) atmospheric chemistry simulations and estimations.

8. The method of claim 1, comprising predicting, for a predetermined time in the future, a methane gas concentration at one of more spatial locations remote from the emission source of the methane gas plume.

9. The method of claim 1, comprising identifying the location of the emission source of the methane gas plume and conducting an operation to terminate leakage of methane gas from the emission source.

10. The method of claim 1, comprising comparing airborne or satellite remote sensing estimates of methane emissions to the terrestrial verification measurements of the methane emissions in the atmospheric boundary layer.

11. The method of claim 1, comprising, in a methane gas mixture, distinguishing methane gas contributions of the methane gas plume from methane gas contributed by one or several other methane gas plumes.

12. The method of claim 1, comprising using the map of estimated methane gas concentrations to determine when a regulatory enforcement action is likely to be taken against an operator responsible for the methane gas plume.

13. The method of claim 1, comprising using the map of estimated methane gas concentrations to determine when an entity responsible for the methane gas plume should enact a voluntary action at the emission source to avoid or to respond to a regulatory enforcement action.

14. A method for quantifying a plume of a fugitive gas, the method comprising the steps of:
detecting a fugitive gas within an emission area;
obtaining weather data for the emission area;
producing a computer-based model for the plume of the fugitive gas;
using the computer-based model for the plume of fugitive gas to determine an emission source of the fugitive gas and/or quantify a volume of the fugitive gas.

15. The method of claim 14, wherein the fugitive gas is selected from CH₂, CO₂, SO2, and CO.

16. The method of claim 14, wherein the step of detecting the fugitive gas within the emission area comprises using a gas sensor selected from the group of trace gas detectors and optical gas imaging cameras.

17. The method of claim 14, wherein the step of detecting the fugitive gas within the emission area comprises determining that the fugitive gas has leaked from the emission source based on measurements at the emission source.

18. The method of claim 14, wherein the step of obtaining weather data for the emission area comprises obtaining the weather data from weather instruments located in the emission area.

19. The method of claim 14, wherein the step of producing the computer-based model for the plume of the fugitive gas comprises using a WRF-GHG model comprising a grid spacing in a range of 5 meters to less than 1 kilometer.

20. The method of claim 14, wherein the step of producing the computer-based model for the plume of the fugitive gas comprises using a WRF-GHG model applied in a large eddy simulation (LES) mode.
